(19) United States
(12) Patent Application Publication
(10) Pub. No.: US 2026/0112483 A1
(43) Pub. Date: Apr. 23, 2026

(54) AUTOMATED CHAT-BASED CARE COORDINATION AGENTS AND ASSISTIVE SYSTEMS

(71) Applicant: Boston Scientific Neuromodulation Corporation, Valencia, CA (US)

(72) Inventors: Dat Thanh Huynh, North Hollywood, CA (US); Matthew Lee McDonald, Glendale, CA (US); Amarpreet Singh Bains, Woodbury, MN (US); Rex James Woon, San Gabriel, CA (US)

(21) Appl. No.: 19/365,929

(22) Filed: Oct. 22, 2025

Related U.S. Application Data
(60) Provisional application No. 63/710,405, filed on Oct. 22, 2024.

(51) Int. Cl. G16H 40/63

(52) U.S. Cl. CPC G16H 40/63

(57) ABSTRACT

Systems and methods can coordinate information among multiple users associated with a neurostimulation device, via an artificial intelligence (AI) system and a conversational bot. An example method for configuring and operating the agent includes: obtaining neurostimulation device data associated with configuration and use of the neurostimulation device; providing the neurostimulation device data as a data source for the AI data processing system that includes a pre-trained large language model (LLM) and an agent to interact with the LLM; configuring the agent with conversation instructions to cause the agent to perform natural language conversations with respective human users based on the configuration and use of the neurostimulation device; and operating the agent to coordinate content of a first natural language conversation that occurs between the agent and a first human user with content of a second natural language conversation that occurs between the agent and a second human user.

### CLAIM OF PRIORITY

This application claims the benefit of U.S. Provisional Application No. 63/710,405, filed on Oct. 22, 2024, which is hereby incorporated by reference in its entirety.

### TECHNICAL FIELD

This document relates generally to data processing obtained in connection with the use of medical devices, and more particularly, to systems, devices, and methods for generating and presenting information associated with an implanted electrical stimulation treatment, including textual interfaces and artificial intelligence operations that assist the operation of neurostimulation treatment devices used for pain treatment, movement disorders, and/or management of such conditions.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). A neurostimulation system can be used to electrically stimulate tissue or nerve centers to treat nervous or muscular disorders. For example, an SCS system may be configured to deliver electrical pulses to a specified region of a patient's spinal cord, such as particular spinal nerve roots or nerve bundles, to produce an analgesic effect that masks pain sensation, or to produce a functional effect that allows increased movement or activity of the patient. Other forms of neurostimulation may include a DBS system which uses similar pulses of electricity at particular locations in the brain to reduce symptoms of essential tremors, Parkinson's disease, psychological disorders, or the like.

To correctly use and optimize a neurostimulation system, patients will at times need support, education, or help. Timely education and support may be difficult for a patient to obtain, due to hard to read directions for use, or the inability for medical care team members (e.g., medical device company representatives (reps), customer care agents, etc.) to provide personalized assistance. For instance, a patient may require education about topics such as how to power on and use their device, charge the device, or may not understand what is happening in their treatment plan, etc. Medical care team members may provide support by providing information about where they are in their treatment journey, providing instructions on how to switch programs to optimize their treatment, etc. The medical care team members may also help resolve patient complaints about the use of the device and device operation, which often need to be handled in a timely and appropriate manner.

The delivery of patient assistance is complicated in many of these situations, because different actors (e.g., medical company representative or patient care assistance team, physicians, caregivers, etc.) need to receive and provide complimentary information in different roles. Coordinating communication between these actors—and the patient—is challenging and often relies on many manual or incomplete approaches.

### SUMMARY

Example 1 is a system to enable coordination of medical information among multiple users associated with a neurostimulation device, with the system comprising: one or more processors; and one or more memory devices comprising instructions, which when executed by the one or more processors, cause the one or more processors to: obtain neurostimulation device data associated with configuration and use of the neurostimulation device; provide the neurostimulation device data as a data source for an artificial intelligence (AI) data processing system, the AI data processing system including a pre-trained model and an agent to interact with the pre-trained model; configure the agent with conversation instructions, the conversation instructions to cause the agent to perform natural language conversations with respective human users based on the configuration and use of the neurostimulation device; and operate the agent to coordinate content of a first natural language conversation that occurs between the agent and a first human user with content of a second natural language conversation that occurs between the agent and a second human user.

Example 2 includes the subject matter of Example 1, optionally further adding subject matter where the pre-trained model includes a large language model (LLM), wherein the first human user is a patient using the neurostimulation device, and wherein the second human user is a user associated with care of the patient.

Example 3 includes the subject matter of Example 2, optionally further adding subject matter where the instructions further cause the one or more processors to: operate the agent to generate recommendations for the patient, wherein the recommendations for the patient relate to: (i) the configuration and use of the neurostimulation device, or (ii) information related to a medical condition to be treated by the neurostimulation device; and output the recommendations in the first natural language conversation occurring between the agent and the patient.

Example 4 includes the subject matter of any one or more of Examples 1-3, optionally further adding subject matter where to coordinate the content of the first natural language conversation with the content of the second natural language conversation includes: conducting the first natural language conversation between the first human user and the agent; identifying a condition for conversation coordination, based on content in the first natural language conversation or the second natural language conversation; conducting the second natural language conversation between the second human user and the agent; and joining the second human user into the first natural language conversation.

Example 5 includes the subject matter of any one or more of Examples 1-4, optionally further adding subject matter where to coordinate the first natural language conversation with the second natural language conversation includes: conducting the first natural language conversation between the first human user and the agent; conducting the second natural language conversation between the first human user and the agent; identifying a condition for conversation coordination, based on content in the first natural language conversation; modifying the first natural language conversation based on content from the second natural language conversation, as managed by the agent; and modifying the second natural language conversation based on content from the first natural language conversation, as managed by the agent.

Example 6 includes the subject matter of any one or more of Examples 1-5, optionally further adding subject matter to: operate the agent to coordinate the content of the first natural language conversation and the content of the second natural language conversation with a third natural language conversation that occurs between the agent and a third human user.

Example 7 includes the subject matter of any one or more of Examples 1-6, optionally further adding subject matter to: identify a special operational condition related to the configuration and use of the neurostimulation device, based on the content of the first natural language conversation or the content of the second natural language conversation; and operate the agent to provide a notification of the special operational condition in the first natural language conversation or the second natural language conversation.

Example 8 includes the subject matter of any one or more of Examples 1-7, optionally further adding subject matter to: obtain patient data associated with a medical condition to be treated by the neurostimulation device; and provide the patient data as another data source for the AI data processing system; wherein the conversation instructions are further to cause the agent to perform the natural language conversations with the respective human users based on the medical condition.

Example 9 includes the subject matter of any one or more of Examples 1-8, optionally further adding subject matter to: maintain historical conversation data of the agent as another data source for the AI data processing system, wherein the historical conversation data is based on previous natural language conversations provided between the respective human users and the agent; wherein the conversation instructions are further to cause the agent to perform the natural language conversations with the respective human users based on the historical conversation data.

Example 10 includes the subject matter of any one or more of Examples 1-9, optionally further adding subject matter to: record a conversation history based on the first natural language conversation occurring between the agent and the first human user and based on the second natural language conversation occurring between the agent and the second human user; and configure the agent to utilize information from the conversation history in subsequent natural language conversations with the first human user and with the second human user.

Example 11 includes the subject matter of any one or more of Examples 1-10, optionally further adding subject matter where the agent is configured to provide the natural language conversations using: an interface for use by a patient device, wherein the patient device is a smartphone operable by a patient; or an interface for use by a medical user device, wherein the medical user device is a computing device operable by a medical user.

Example 12 includes the subject matter of any one or more of Examples 1-11, optionally further adding subject matter to: identify a programming change related to the configuration and use of the neurostimulation device, based on the content of the first natural language conversation or the content of the second natural language conversation; and transmit a command, based on the identified programming change, to reconfigure one or more programming data values of the neurostimulation device.

Example 13 includes the subject matter of Example 12, optionally further adding subject matter where the command to reconfigure the one or more programming data values of the neurostimulation device causes a change to one or more of: timing, amplitude, frequency, intensity, duration, pulse patterns, pulse shapes, a spatial location of pulses, waveform shapes, or a spatial location of waveform shapes, of modulated energy provided with a plurality of leads of the neurostimulation device.

Example 14 is a machine-readable medium including instructions, which when executed by a machine, cause the machine to perform the operations of the system of any of the Examples 1 to 13.

Example 15 is a method to perform the operations of the system of any of the Examples 1 to 13.

Example 16 is a method for enabling coordination of medical information among multiple users associated with a neurostimulation device, comprising: obtaining neurostimulation device data associated with configuration and use of the neurostimulation device; providing the neurostimulation device data as a data source for an artificial intelligence (AI) data processing system, the AI data processing system including a pre-trained model and an agent to interact with the pre-trained model; configuring the agent with conversation instructions, the conversation instructions to cause the agent to perform natural language conversations with respective human users based on the configuration and use of the neurostimulation device; and operating the agent to coordinate content of a first natural language conversation that occurs between the agent and a first human user with content of a second natural language conversation that occurs between the agent and a second human user.

Example 17 includes the subject matter of Example 16, optionally further adding subject matter where the pre-trained model includes a large language model (LLM), wherein the first human user is a patient using the neurostimulation device, and wherein the second human user is a user associated with care of the patient.

Example 18 includes the subject matter of Example 17, optionally further adding subject matter including operating the agent to generate recommendations for the patient, wherein the recommendations for the patient relate to: (i) the configuration and use of the neurostimulation device, or (ii) information related to a medical condition to be treated by the neurostimulation device; and outputting the recommendations in the first natural language conversation occurring between the agent and the patient.

Example 19 includes the subject matter of any one or more of Examples 16-18, optionally further adding subject matter where to coordinate the content of the first natural language conversation with the content of the second natural language conversation includes: conducting the first natural language conversation between the first human user and the agent; identifying a condition for conversation coordination, based on content in the first natural language conversation or the second natural language conversation; conducting the second natural language conversation between the second human user and the agent; and joining the second human user into the first natural language conversation.

Example 20 includes the subject matter of any one or more of Examples 16-19, optionally further adding subject matter where to coordinate the first natural language conversation with the second natural language conversation includes: conducting the first natural language conversation between the first human user and the agent; conducting the second natural language conversation between the first human user and the agent; identifying a condition for conversation coordination, based on content in the first natural language conversation; modifying the first natural language conversation based on content from the second natural language conversation, as managed by the agent; and modifying the second natural language conversation based on content from the first natural language conversation, as managed by the agent.

Example 21 includes the subject matter of any one or more of Examples 16-20, optionally further adding subject matter including: operating the agent to coordinate the content of the first natural language conversation and the content of the second natural language conversation with a third natural language conversation that occurs between the agent and a third human user.

Example 22 includes the subject matter of any one or more of Examples 16-21, optionally further adding subject matter including: identifying a special operational condition related to the configuration and use of the neurostimulation device, based on the content of the first natural language conversation or the content of the second natural language conversation; and operating the agent to provide a notification of the special operational condition in the first natural language conversation or the second natural language conversation.

Example 23 includes the subject matter of any one or more of Examples 16-22, optionally further adding subject matter including: obtaining patient data associated with a medical condition to be treated by the neurostimulation device; and providing the patient data as another data source for the AI data processing system; wherein the conversation instructions are further to cause the agent to perform the natural language conversations with the respective human users based on the medical condition.

Example 24 includes the subject matter of any one or more of Examples 16-23, optionally further adding subject matter including: maintaining historical conversation data of the agent as another data source for the AI data processing system, wherein the historical conversation data is based on previous natural language conversations provided between the respective human users and the agent; wherein the conversation instructions are further to cause the agent to perform the natural language conversations with the respective human users based on the historical conversation data.

Example 25 includes the subject matter of any one or more of Examples 16-24, optionally further adding subject matter including: identifying a programming change related to the configuration and use of the neurostimulation device, based on the content of the first natural language conversation or the content of the second natural language conversation; and transmitting a command, based on the identified programming change, to reconfigure one or more programming data values of the neurostimulation device; wherein the command to reconfigure the one or more programming data values of the neurostimulation device causes a change to one or more of: timing, amplitude, frequency, intensity, duration, pulse patterns, pulse shapes, a spatial location of pulses, waveform shapes, or a spatial location of waveform shapes, of modulated energy provided with a plurality of leads of the neurostimulation device.

Example 26 is a computing system to enable coordination of medical information among multiple users associated with a neurostimulation device, the computing system comprising: one or more processors; and one or more memory devices comprising instructions, which when executed by the one or more processors, cause the one or more processors to: obtain neurostimulation device data associated with configuration and use of the neurostimulation device; provide the neurostimulation device data as a data source for an artificial intelligence (AI) data processing system, the AI data processing system including a pre-trained model and an agent to interact with the pre-trained model; configure the agent with conversation instructions, the conversation instructions to cause the agent to perform natural language conversations with respective human users based on the configuration and use of the neurostimulation device; and operate the agent to coordinate content of a first natural language conversation that occurs between the agent and a first human user with content of a second natural language conversation that occurs between the agent and a second human user.

Example 27 includes the subject matter of Example 26, optionally further adding subject matter where the pre-trained model includes a large language model (LLM), wherein the first human user is a patient using the neurostimulation device, and wherein the second human user is a user associated with care of the patient.

Example 28 includes the subject matter of Example 27, optionally further includes subject matter to operate the agent to generate recommendations for the patient, wherein the recommendations for the patient relate to: (i) the configuration and use of the neurostimulation device, or (ii) information related to a medical condition to be treated by the neurostimulation device; and output the recommendations in the first natural language conversation occurring between the agent and the patient.

Example 29 includes the subject matter of any one or more of Examples 26-28, optionally further adding subject matter to coordinate the content of the first natural language conversation with the content of the second natural language conversation by: conducting the first natural language conversation between the first human user and the agent; identifying a condition for conversation coordination, based on content in the first natural language conversation or the second natural language conversation; conducting the second natural language conversation between the second human user and the agent; and joining the second human user into the first natural language conversation.

Example 30 includes the subject matter of any one or more of Examples 26-29, optionally further adding subject matter to coordinate the first natural language conversation with the second natural language conversation includes: conducting the first natural language conversation between the first human user and the agent; conducting the second natural language conversation between the first human user and the agent; identifying a condition for conversation coordination, based on content in the first natural language conversation; modifying the first natural language conversation based on content from the second natural language conversation, as managed by the agent; and modifying the second natural language conversation based on content from the first natural language conversation, as managed by the agent.

Example 31 includes the subject matter of any one or more of Examples 26-30, optionally further adding subject matter to: operate the agent to coordinate the content of the first natural language conversation and the content of the second natural language conversation with a third natural language conversation that occurs between the agent and a third human user.

Example 32 includes the subject matter of any one or more of Examples 26-31, optionally further adding subject matter to: identify a special operational condition related to the configuration and use of the neurostimulation device, based on the content of the first natural language conversation or the content of the second natural language conversation; and operate the agent to provide a notification of the special operational condition in the first natural language conversation or the second natural language conversation.

Example 33 includes the subject matter of any one or more of Examples 26-32, optionally further adding subject matter to: obtain patient data associated with a medical condition to be treated with the neurostimulation device; and provide the patient data as another data source for the AI data processing system; wherein the conversation instructions are further to cause the agent to perform the natural language conversations with the respective human users based on the medical condition.

Example 34 includes the subject matter of any one or more of Examples 26-33, optionally further adding subject matter to: maintain historical conversation data of the agent as another data source for the AI data processing system, wherein the historical conversation data is based on previous natural language conversations provided between the respective human users and the agent; wherein the conversation instructions are further to cause the agent to perform the natural language conversations with the respective human users based on the historical conversation data.

Example 35 includes the subject matter of any one or more of Examples 26-34, optionally further adding subject matter to: identify a programming change related to the configuration and use of the neurostimulation device, based on the content of the first natural language conversation or the content of the second natural language conversation; and transmit a command, based on an identified programming change, to reconfigure one or more programming data values of the neurostimulation device; wherein the command to reconfigure the one or more programming data values of the neurostimulation device causes a change to one or more of: timing, amplitude, frequency, intensity, duration, pulse patterns, pulse shapes, a spatial location of pulses, waveform shapes, or a spatial location of waveform shapes, of modulated energy provided with a plurality of leads of the neurostimulation device.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates a data coordination system to analyze data and generate coordinated interactions in connection with the use of neurostimulation device usage, according to an example.

FIG. 2 illustrates a neuromodulation system as an example of a medical device system, according to an example.

FIG. 3 further illustrates a neuromodulation system as an ambulatory medical device, such as implemented by a neurostimulation treatment, according to an example.

FIG. 4 illustrates a neuromodulation device connected to a programming device, according to an example.

FIG. 5 illustrates data interactions with a care coordination system, for coordination of chat sessions in connection with a neuromodulation device use and programming, according to an example.

FIG. 6 depicts a scenario for providing natural language conversations between a patient user and a medical user using a care coordination system, according to an example.

FIG. 7 depicts user-based data flows, using a care coordination system that includes an artificial intelligence (AI)-based conversational agent, according to an example.

FIG. 8A and FIG. 8B depict user chat sessions and a corresponding flowchart of operations with a care coordination conversational agent, according to an example.

FIG. 9 depicts agent-coordinated data flows, using a care coordination system that includes an artificial intelligence (AI)-based conversational agent, according to an example.

FIG. 10A and FIG. 10B depict additional agent chat sessions and a corresponding flowchart of operations with a care coordination conversational agent, according to an example.

FIG. 11 illustrates a data processing flow for implementing programming changes for neurostimulation treatments in a human patient, in connection with a care coordination system, according to an example.

FIG. 12 illustrates a processing method implemented by a system or device to enable coordination of medical information among multiple users associated with a neurostimulation device, according to an example.

FIG. 13 illustrates, by way of example, a block diagram of an embodiment of a system (e.g., a computing system) for the exchange of medical information, in connection with a care coordination system, according to an example.

FIG. 14 illustrates, by way of example, a block diagram of an embodiment of a system (e.g., a computing system) implementing neurostimulation programming circuitry to cause programming of an implantable electrical neurostimulation device, according to an example.

FIG. 15 is a block diagram illustrating a machine in the example form of a computer system, within which a set or sequence of instructions may be executed to cause the machine to perform any one of the methodologies discussed herein, according to an example.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Various embodiments of the present subject matter relate to user interfaces, data algorithms and models, data processing systems, workflows, and computing systems and devices used in connection with the care coordination for patient use of a neurostimulation device. As an example, aspects include how to generate, deliver, track, and evaluate content relating to neurostimulation device assistance and programming, coordinated among the patient, the patient's medical care team, and other relevant actors.

A care coordination system is described that system gathers and integrates information about the patient and their neurostimulation treatment and device from multiple sources. For example, the care coordination system obtains and uses information about the patient's support natural language questions and conversations, data measurements from an inferred or stated health state, responses to validated questionnaires, and/or natural language conversation history. The care coordination system is also capable to obtain and use information from other sources of medical data, such as device data from the implantable program generator (IPG), patient medical records and medical history relating to the medical condition being treated and related medical conditions, sensor data from wearables, etc.

The care coordination system extracts pertinent information from this medical data and processes this information within an AI model. For example, the care coordination system can operate Large Language Models (LLMs) and other AI-based trained models—in addition to natural language processing (NLP) algorithms—that extract meaning from existing natural language conversations and generate new natural language content. The care coordination system analyzes the information and makes rules-or AI-based decisions on what care coordination action to take next. The care coordination system may generate and send automated messages to the patient, provide therapy updates to the device (e.g. turning on a different program or changing program settings), summarize the context, and bring in a human (rep, physician, caregiver, etc.) to continue the conversation, offer advice for next steps, oversee recommendations and approve suggested actions, etc.

A variety of actions thus may be enabled or suggested by the care coordination system. The action needed from the human can vary depending on the risk/certainty level estimated by the system. For instance, in some contexts, this may simply include the review and/or approval of some instructions or directions in a natural language response generated by the system. In other contexts, this may include a full handover (escalation) of the conversation to a human to continue. The care coordination system provides the capability to evaluate and escalate a chat session to a human user based on various factors, such as an algorithmically-estimated potential for risk to patient safety, or inability to generate a response with high confidence. In other examples, a human user can receive a notification, and can selectively join or intervene in the conversation.

Accordingly, the care coordination system may be used for support of a variety of neurostimulation programming deployments, including but not limited to closed-loop and partially-closed-loop programming approaches. The care coordination system may also be used to support or monitor various changes to device programming schedules or settings (e.g., amplitude, duration, timing, and frequency values). The care coordination system manages the conversation, facilitating the conversation and actions in a patient-specific and therapy-specific way; while enabling control and monitoring so that an overseeing human user can see what the system is doing (and override it if needed).

The following describes a “data collection platform”, “data coordination system”, and “data service” that generally refers to portions of a compute platform (e.g., a combination of hardware, firmware and software) with a set of capabilities for collecting, processing, and generating data in connection with natural language conversations and programming related to neurostimulation. A compute platform may be a single platform (e.g., at a single cloud service) or may be organized as more than one platform (e.g., among multiple distributed computing locations) that are configured to cooperate with each other. A compute platform may obtain data from one device or from more than one device. Thus, a therapy device such as an implanted neuromodulation device may provide some portion of the collected data, and a user device (e.g., smartphone) with an interactive feedback user interface (e.g., provided by a smartphone app) may provide another portion of the collected data. A compute platform may also obtain data from other sensor(s) and other data source(s), and be guided by (or under the control of) a clinician or an agent of a device manufacturer.

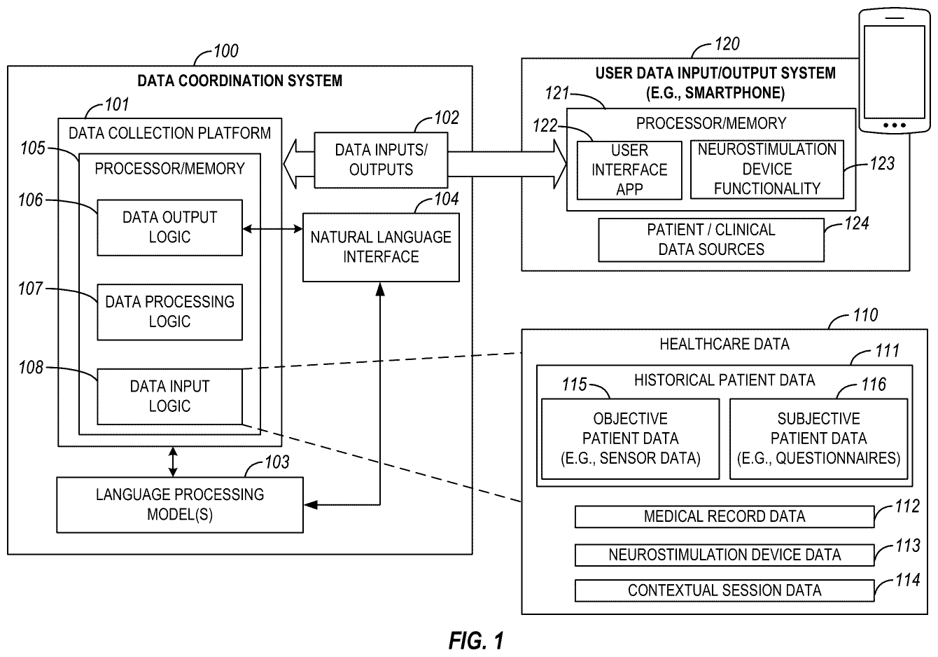

FIG. 1 illustrates, by way of example, an embodiment of a data coordination system 100 configured to analyze data and generate content in connection with the use of neurostimulation conversations and device programming. The illustrated data coordination system 100 is configured to include at least one data collection platform 101, which operates to collect and provide data inputs/outputs 102. The data collection platform 101 is configured to process (e.g., filter, extract, transform) the input data, to produce or evaluate content in natural language conversations relevant to the neurostimulation device use and programming activities. These natural language conversations include agent content that is generated by one or more language processing models 103, such as large language models (LLMs) and trained AI models that can understand and generate human-understandable text.

The language processing models 103 are used to generate various conversational content to be output to a user via a natural language interface 104 (such as a chat interface, a voice conversation interface, etc.). The user may be the patient, a medical user, a clinician, or a combination of these users. In addition to information that is output via the natural language interface, the data collection platform 101 can also provide output data in the form of instructions, recommendations, and controls that are relevant to programming activities, and the collection of feedback or other inputs.

The data coordination system 100 may be implemented at one or more server(s) or other systems remotely located from the patient. The data coordination system 100 may use various network protocols to communicate and transfer data through one or more networks such as the Internet. The data coordination system 100 and data collection platform 101 may include at least one processor configured to execute instructions stored in memory (e.g., depicted as processor(s)/memory 105) to generate data outputs 106, to obtain or evaluate data inputs 108, and to perform data processing 107 on both inputs and outputs and accompanying data. For instance, the data inputs 108 may be selected from a larger set of medical data (such as medical records), to provide specific inputs into the language processing models to generate customized content for a particular human user, to identify a recommended programming change or setting based on historical events or actual patient activities, and to consider the patient's overall context of neurostimulation use and treatment.

The data inputs 108 may include information obtained from a human user directly (e.g., from patient or medical user conversations), or may include different types of healthcare data 110 associated with the patient or the treatment. Examples of healthcare data 110 may include patient data 111, medical record data 112, neurostimulation device data 113, contextual session data 114, or various combinations thereof. The patient data 111 may include objective data 115 such as data collected from physiological sensor(s) and subjective data 116 such as data collected from user question(s) and answers(s) (e.g., “How do you rate your pain?”). Objective and subjective data may be provided by the patient, a caregiver, a clinician, or a third party (device manufacturer or service provider). Objective data, as used herein, is data that can obtained from a measurement or direct observation. Objective data may be measured by a sensor and may be provided via user input when the user has access to objectively determined information. Categories of objective data may include physiological parameter data, therapy data, device data, and environmental data. By way of example and not limitation, physical parameter data may include data such as: heart rate, blood pressure, respiration rate, activity, posture, electromyograms (EMGs), neural responses such as evoked compound action potentials (ecaps), glucose measurements, oxygen levels body temperature, oxygen saturation and gait. By way of example and not limitation, therapy data may include: neuromodulation programs, therapy on/off schedule, dosing, neuromodulation parameters such as waveform, frequency, amplitude, pulse width, period, therapy usage and therapy type. By way of example and not limitation, device data may include: battery information (voltage, charge state, charging history if rechargeable), impedance data, faults, device model, lead models, MRI status, Bluetooth connection logs, connection history with Clinician's Programmer (CP). By way of example and not limitation, environmental data may include: temperature, air quality, pressure, location, altitude, sunny, cloudy, precipitation, etc. Subjective data can include information received from the patient or another human user (e.g., caregiver, clinician, etc.). For example, the patient's quantification of pain can provide subjective data. Subjective data may generally involve user-inputted data. Examples of subjective data include questions with free text answers, multiple choice questions, question tree(s), and different question subject(s). Other data may be stored and/or transferred, including detected event data to track events (e.g., that trigger a response, change data resolution), contextual data, time data, and the like. The event(s), context(s) and time may be detected by the system or may be provided via user input.

The user data input/output system 120 may be implemented at one or more devices located at or operated by a user (such as a patient, medical user, or clinician), via a smartphone, personal computer, tablet, smart home device, a remote programmer, a programming device, or another compute device or platform capable of collecting input and providing output (e.g., the output of text or voice conversations and responses). The user data input/output system 120 may include at least one processor configured to execute instructions stored in memory (e.g., depicted as processor(s)/memory 121) to provide data input(s) and outputs to enable interacting with a conversational agent of the data coordination system 100, and to perform control of related neurostimulation device programming. One such example is a user interface application 122 implemented as a graphical user interface (GUI), which provides commands to enable neurostimulation device functionality 123 such as specialized programming changes or modifications.

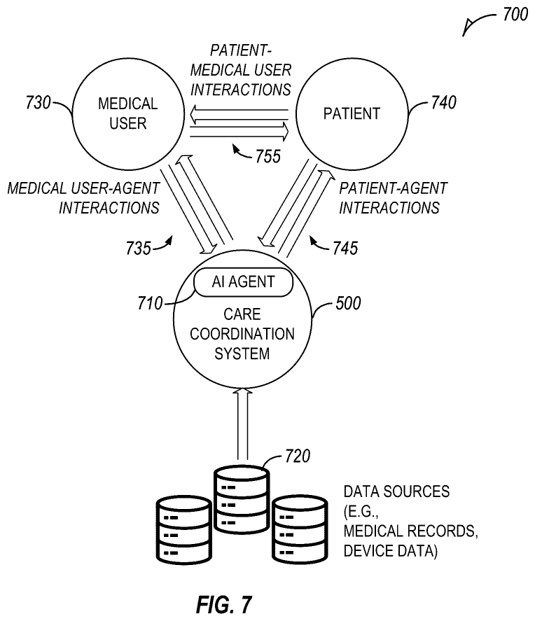

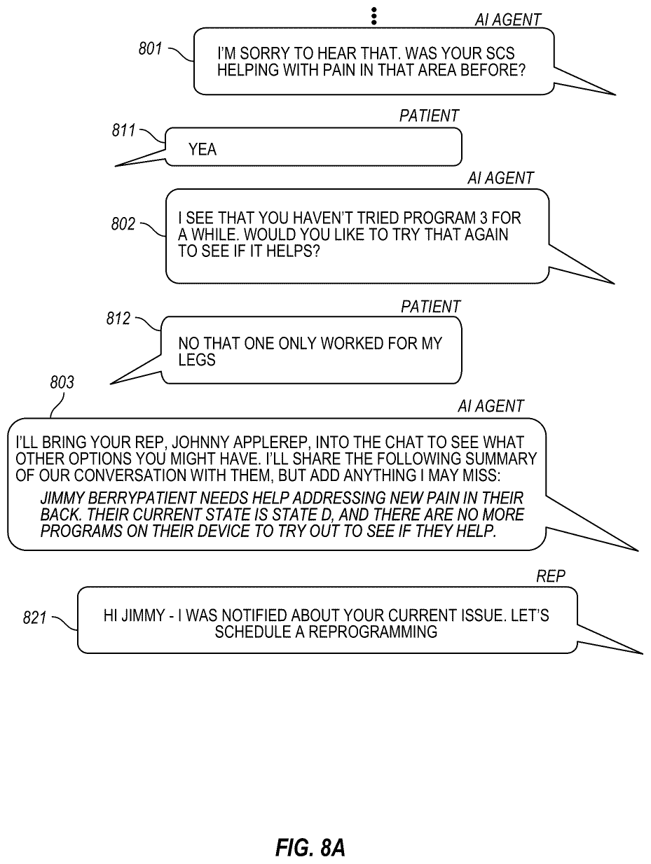

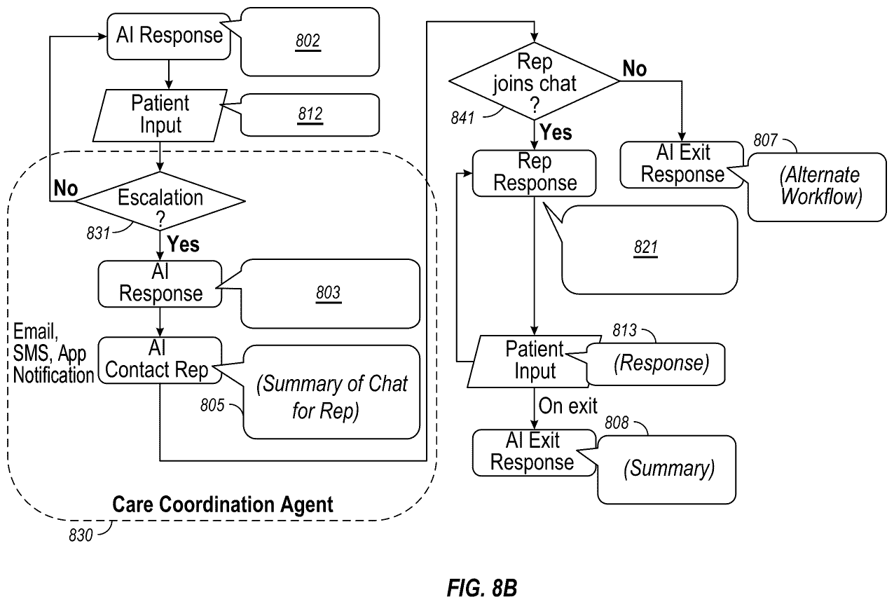

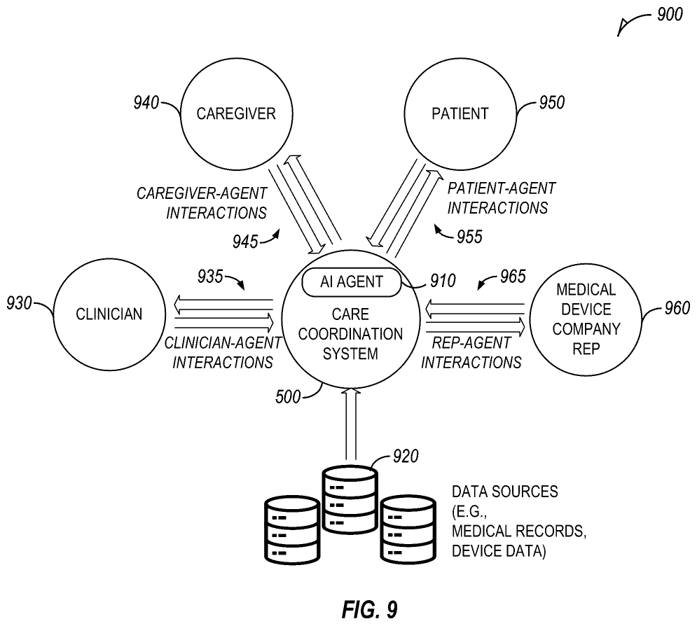

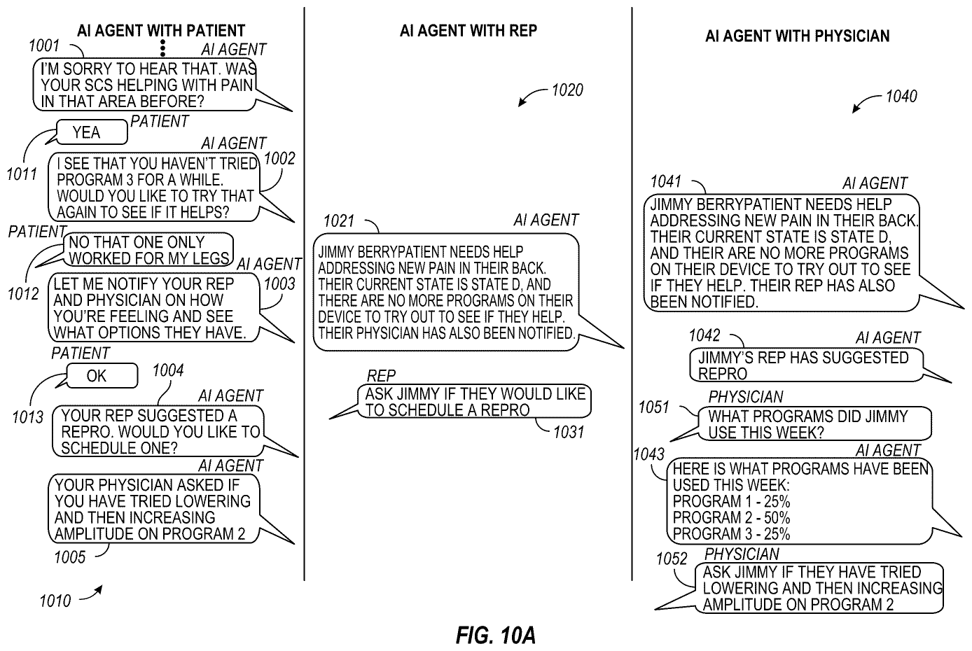

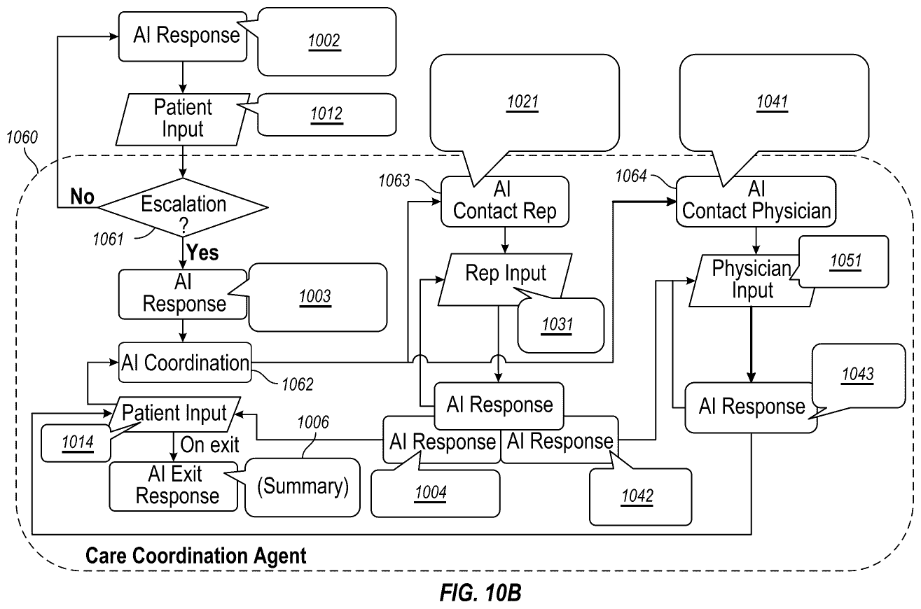

Examples of workflows for user natural language conversations (and the types of data inputs, data outputs, and conditions) are illustrated in FIGS. 7-8B for scenarios where a patient can directly communicate with a care coordination system or with another medical user; and are illustrated in FIGS. 9-10B for scenarios where users can communicate with one another via a care coordination system.

The user data input/output system 120 may obtain information from patient or clinical data sources, including but not limited to data from physiological sensors that can detect patient state information (e.g., activity, sleep), event(s), indicators of device usage, patient compliance with data collection and/or therapy, etc. The user data input/output system 120 may directly or indirectly capture measurements from the patient or clinical data sources, and from associated external systems. Examples of external system(s) include remote controls, programmers, phones, tablets, smart watches, personal computers, and the like. In some examples, the external system may be configured to provide the medical record data 112, the neurostimulation device data 113, or the contextual session data 114 for use by the data coordination system 100.

The language processing model 103 may operate one or more large language models (LLMs) or trained artificial intelligence models (such as a neural network) that has been trained to infer or generate human-understandable text or graphical content (e.g., images, video, audio, etc.) One or multiple instances of a model may be patient-specific or population-based models. The language processing model 103 may consider multiple aspects (and, when appropriate, dynamic aspects) of patient-specific or population data such as the healthcare data 110, sensor data, and rules and information from a variety of data sources.

In some examples, the data collection platform 101 and the data coordination system 100 may directly interface with one or more medical device(s), external system(s) or other healthcare related data source(s) to collect the healthcare data 110. One or more of the medical device(s), external system(s) or other healthcare-related data source(s) may include technology used by the data coordination system 100 to collect data, and thus may form part of the data collection platform 101. Examples of medical devices include implantable and wearable devices. The medical device may be configured to only collect data, to only deliver therapy, or to both collect data and deliver therapy. Thus, the medical device may provide the patient data 111, the medical record data 112, the neurostimulation device data 113, and contextual session data 114, particularly in specialized neurostimulation programming environments.

Other healthcare-related data source(s) for the medical record data 112 may include patient data received via a provider's server that stores patient health records (e.g., test results, doctor notes, prescriptions, and the like). Other healthcare-related data sources may include apps on a patient's smartphone or other computing device, or the data on a server accessed by those apps. By way of example and not limitation, this type of data may include heart rate, blood pressure, weight, and the like collected by the patient in their home.

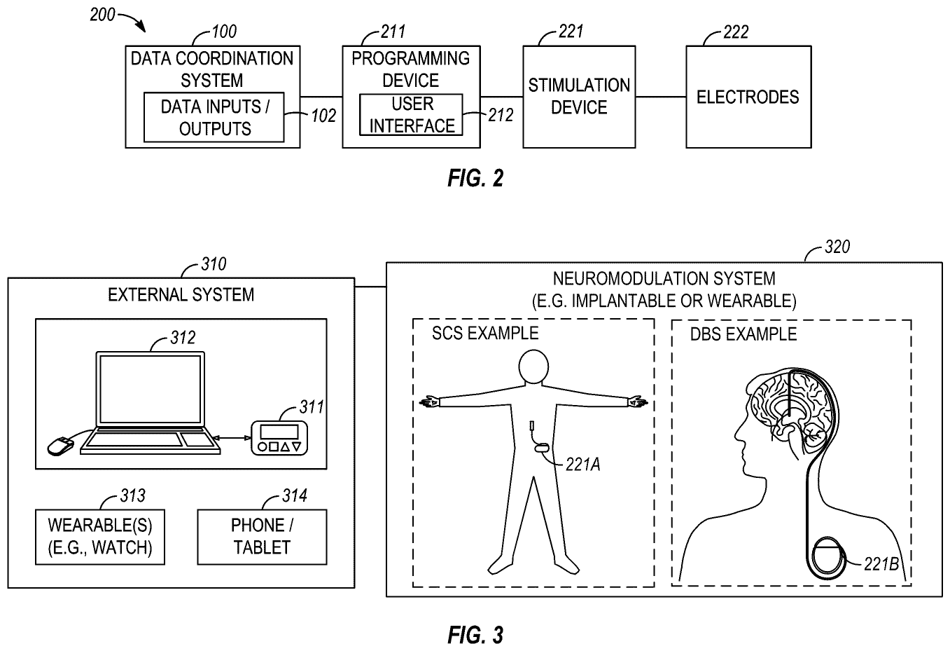

FIG. 2 illustrates a neuromodulation system as an example of a medical device system. The medical device may be configured, by way of example and not limitation, to deliver an electrical therapy using one or more electrodes 222 provided by a stimulation device 221. In the illustrated embodiment, the medical device may be a neurostimulation device, and the system may be a neuromodulation system 200.

The illustrated neuromodulation system 200 includes electrodes 222, the stimulation device 221 and a programming system such as a programming device 211. The programming system may include multiple devices. The electrodes 222 are configured to be placed on or near one or more neural targets in a patient. The stimulation device 221 is configured to be electrically connected to the electrodes 222 and deliver neuromodulation energy, such as in the form of electrical pulses, to the one or more neural targets though the electrodes 222. The system may also include sensing circuitry to sense a physiological signal, which may but does not necessarily form a part of stimulation device 221. The delivery of the neuromodulation is controlled using a plurality of modulation parameters that may specify the electrical waveform (e.g., pulses or pulse patterns or other waveform shapes) and a selection of electrodes through which the electrical waveform is delivered. In various embodiments, at least some parameters of the plurality of modulation parameters are programmable by a user, such as a physician or other caregiver, using one or more programs. The programming device 211 thus provides the user with accessibility to the user-programmable parameters. The programming device 211 may also provide the use with data indicative of the sensed physiological signal or feature(s) of the sensed physiological signal.

In various embodiments, the programming device 211 is configured to be communicatively coupled to the stimulation device 221 via a wired or wireless link. In various embodiments, the programming device 211 includes a user interface 212 such as a graphical user interface (GUI) that allows the user to set and/or adjust values of the user-programmable modulation parameters, including parameters provided via recommendations as discussed herein. The user interface 212 may also allow the user to view the data indicative of the sensed physiological signal or feature(s) of the sensed physiological signal and may allow the user to compare the sensed data to expected or recommended data values. The data may be provided to the data coordination system 100, which processes data inputs and outputs 102 to assist the user with the operation, training, configuration, maintenance, or improvement of the stimulation device 221.

The user interface 212 of the programming device 211 may be used to allow the user to answer questions to provide healthcare-related data, although other devices (such as a patient smartphone) may be used as discussed below. The user interface 212 of the programming device 211 may also include the ability to receive or display recommendations, such as recommendations downloaded from a network from other local devices (such as a patient smartphone). Therapy parameters, programming selection, electrode selection, and other operational parameters entered or selected in the user interface 212 may also provide data as an input to the data coordination system 100. Additional sensor(s) may also provide data for use by the data coordination system 100.

FIG. 3 illustrates, by way of example and not limitation, the neurostimulation system of FIG. 2 implemented in a spinal cord stimulation (SCS) system or a deep brain stimulation (DBS) system. The illustrated neuromodulation system 320 connects with an external system 310 that may include at least one programming device. The illustrated external system 310 may include a programmer 312 (e.g., clinician programmer) configured for use by a clinician to communicate with and program the neurostimulator, and a remote control device 311 configured for use by the patient to communicate with and program the neurostimulator. For example, the remote control device 311 may allow the patient to turn a therapy on and off and/or may allow the patient to adjust patient-programmable parameter(s) of the plurality of modulation parameters (e.g., by switching programs).

FIG. 3 further illustrates the neuromodulation system 320 as an ambulatory medical device, such as implemented by stimulation device 221A or stimulation device 221B. Examples of ambulatory devices include wearable or implantable neuromodulators. The external system 310 may include a network of computers, including computer(s) remotely located from the ambulatory medical device that are capable of communicating via one or more communication networks with the programmer 312 and/or the remote control device 311. The remotely located computer(s) and the ambulatory medical device may be configured to communicate with each other via another external device such as the programmer 312 or the remote control device 311.

The external system 310 may also include one or more wearables 313 and a portable device 314 such as a smartphone or tablet. In some examples, the wearables 313 and the portable device 314 may allow a user to obtain and provide input data, such as sensor data values (e.g., from a physiologic sensor of a wearable) or feedback/status information (e.g., on a phone/tablet screen) in connection with a data collection process. In some examples, the remote control device 311 and/or the programmer 312 also may display recommendations or program settings derived from recommendations as part of a programming process. The remote control device 311 and/or the programmer 312 may be used to communicate other aspects of input and output, including inputs from the usage data of various neurostimulation programs, events associated with such programs, and the like.

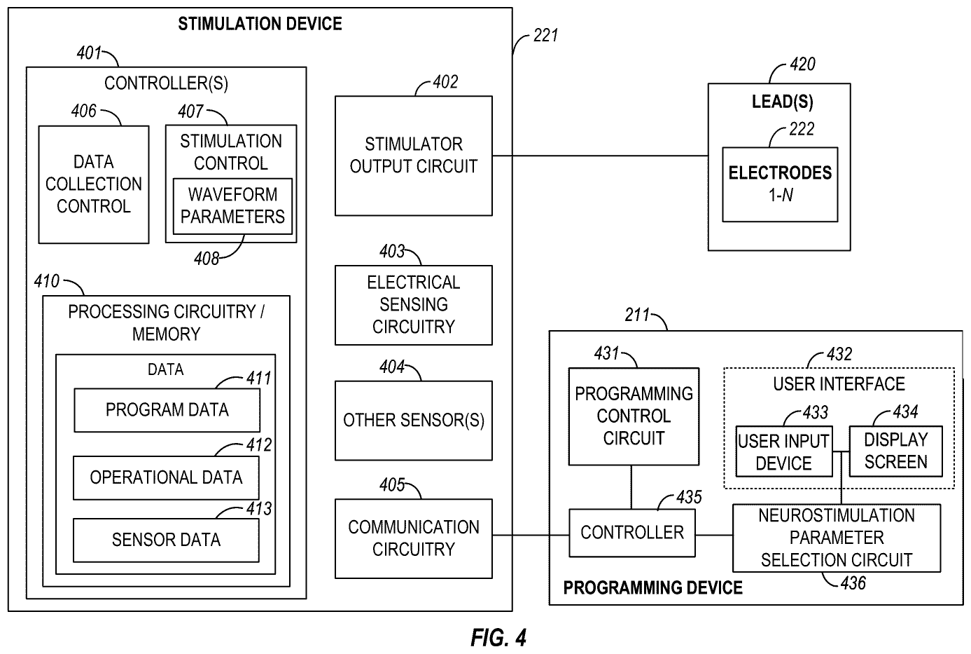

FIG. 4 illustrates, by way of example, an embodiment of a neuromodulation device, such as may be implemented as the stimulation device 221 illustrated in FIGS. 2 and 3. The stimulation device 221 may be configured to be connected to electrode(s) 222, illustrated as N electrodes, via one or more leads 420. Any one or more of the electrodes 222 may be configured for use to deliver modulation energy, sense electrical activity, or both deliver modulation energy and sense electrical activity. The stimulation device 221 may include a stimulator output circuit 402 configured to deliver modulation energy to electrode(s). The stimulator output circuit 402 may be configured with multiple (e.g., two or more) channels for delivering modulation energy, where each channel may be independently controlled with respect to other channel(s). For example, the stimulator output circuit 402 may have independent sources such as independent current sources or independent voltage sources.

In various examples, the electrodes 222 may include a stimulation electrode or a sensing electrode. The stimulation electrode is configured for use in delivering modulation energy, and the sensing electrode is configured for use in sensing electrical activity. As illustrated, the stimulation electrode may also be used in sensing electrical activity, and the sensing electrode may also be used in delivering modulation energy. Thus, the term “stimulation electrode” does not necessarily exclude the electrode from also being used to sense electrical activity; and the term “sensing electrode” does not necessarily exclude the electrode from also being used to deliver modulation energy.

The stimulation device 221 may include electrical sensing circuitry 403 configured to receive sensed electrical energy from the electrode(s), such as may be used to sense electrical activity in neural tissue or muscle tissue. The sensing circuitry may be configured to process signals in multiple (e.g., two or more) channels. By way of example and not limitation, the electrical sensing circuitry 403 may be configured to amplify and filter the signal(s) in the channel(s).

The controller 401 may be configured to detect one or more features in the sensed signals. Examples of features that may be detected include peaks (e.g., minimum and/or maximum peaks including local peaks/inflections), range between minimum/maximum peaks, local minima and/or local maxima, area under the curve (AUC), curve length between points in the curve, oscillation frequency, rate of decay after a peak, a difference between features, and a feature change with respect to a baseline. Detected feature(s) may be fed into a control algorithm, which may use relationship(s) between the feature(s) and waveform parameter(s) to determine feedback for control of the therapy. Some embodiments of the stimulation device 221 may include or be configured to receive data from other sensor(s) 404. The other sensor(s) 404 may include physiological sensor(s), environmental sensor(s), or proximity sensor(s).

The stimulation device 221 may include a controller 401 operably connected to the stimulator output circuit 402 and the electrical sensing circuitry 403. The controller 401 may include a stimulation control 407 (e.g., logic) configured for controlling the stimulator output circuit 402. For example, the stimulation control 407 may include start/stop information for the stimulation and/or may include relative timing information between stimulation channels. The stimulation control 407 may include waveform parameters 408 (e.g., associated with a program or a defined set of parameters) that control the waveform characteristics of the waveform produced by the stimulator output circuit 402. The waveform parameters 408 may include, by way of example and not limitation, amplitude, frequency, and pulse width parameters. The waveform parameters 408 may include, by way of example and not limitation, regular patterns such as patterns regularly repeat with same pulse-to-pulse interval and./or irregular patterns of pulses such as patterns with variable pulse-to-pulse intervals. The waveform parameters may, but do not necessary, define more than one waveform shape (e.g., including a shape other than square pulses with different widths or amplitudes). The stimulation control 407 may be configured to change waveform parameter(s) (e.g., one or more waveform parameters) in response to user input and/or automatically in response to feedback.

The controller 401 may include a data collection control 406 configured for use by the stimulation device 221, and the data collection platform 101 of a data coordination system 100 (see FIGS. 1-2), to collect healthcare-related data. The controller 401 may include a processor and/or memory 410 (e.g., with instructions) for use to control the data collection using the data collection control 406 and control the stimulation via the stimulation control 407. The memory may also provide storage for storing different types of collected healthcare-related data, such as program data 411, operational data 412, sensor data 413, and the like. The program data 411 and operational data 412 may be provided or implemented as a result of the programming recommendations or changes provided with the techniques discussed herein. Examples of sensor data 413 collected by the stimulation device 221 or other devices discussed herein may include, by way of example and not limitation, heart rate, heart rate variability, oxygen saturation, activity, posture, steps, gait, temperature, evoked compound action potentials (ECAPS), electromyograms (EMGs), electroencephalograms (EEGs), weight, blood pressure, and the like. Examples of program data 411 may include, by way of example and not limitation, stimulation settings such as amplitude, pulse width, pulse frequency period, duration of burst of pulses, active electrodes, electrode fractionalization controlling the distribution of energy (e.g., current) to active electrodes, waveforms, pulse patterns including various complex patterns, and the like. Examples of operational data 412 of the stimulation device 221 may include, by way of example and not limitation, electrode-tissue impedance, fault conditions, battery information such as battery health, battery life, voltage, charge state, charging history if rechargeable, MRI status, Bluetooth connection logs, connection with a clinician programmer, hours of operation/duration of implant, and the like. Other device information may include device model and lead model.

The neuromodulation device may include communication circuitry 405 configured for use to communicate with other device(s) such as a programming device 211, remote control, phone, tablet and the like. The healthcare-related data may be transferred from the neuromodulation device to the data coordination system 100, as discussed above. As shown, a programming device 211 includes a programming control circuit 431, a user interface 432 (e.g., including a user input device 433 such as buttons and a display screen 434), a controller 435, and other components (e.g., an external communication device, not shown) to effect programming of a connected neurostimulation device. The operation of the neurostimulation parameter selection circuit 436 enables the selection, modification, and implementation of a particular set of parameters or settings for neurostimulation programming (e.g., via selection of a program, implementation of a closed-loop or open-loop programming process, specification by a patient or clinician, or the like). As used herein, a “partially closed-loop” system refers to use of a closed-loop system that automatically generates or suggests programming values, but also relies on some form of clinician, patient, or third party intervention that customizes the settings, timing, or use of the programming values. This may include intervention provided in connection with natural language conversations and care coordination among multiple users.

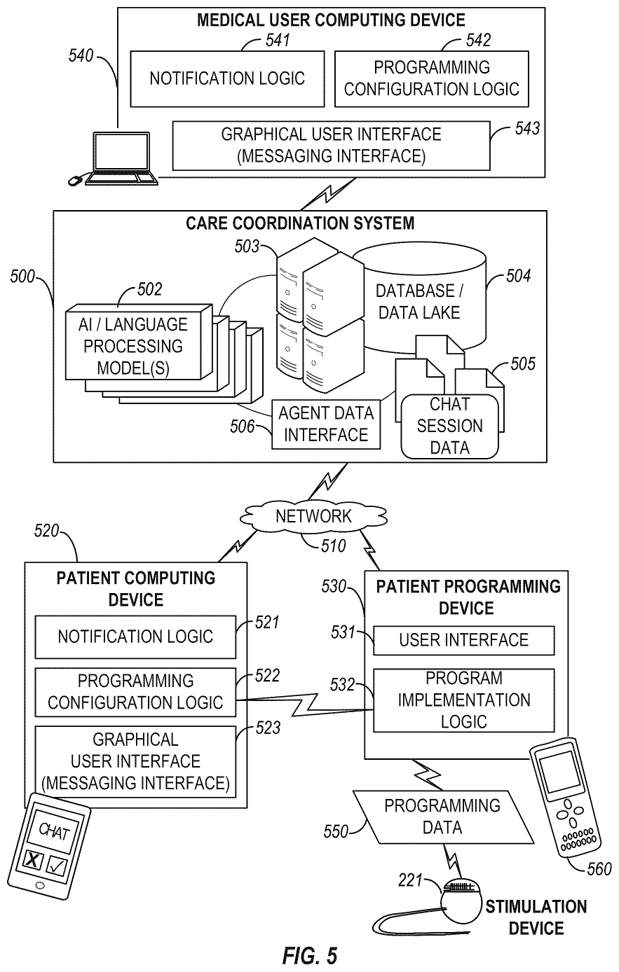

FIG. 5 illustrates, by way of example, an embodiment of data interactions with a care coordination system 500, used for coordination of support for neurostimulation use and programming with a stimulation device 221. The care coordination system 500 in this example provides an implementation of the data coordination system 100 previously discussed. This figure illustrates two devices used by a patient, e.g., a patient computing device 520 and a patient programming device 530; and this figure also illustrates one device used by a medical user who assists the patient (e.g., clinician, company representative, assistance agent). However, additional users and devices may be integrated with use of the care coordination system 500.

The care coordination system 500 includes computer hardware 503 such as servers, processing circuitry, AI accelerators, etc. At a high level, the care coordination system 500 executes a trained model with the computer hardware 503 and processes relevant data as part of input to the model. The trained model may be provided by one of more AI/language processing models 502—such as an LLM—to generate natural language in conversations with patient users and medical users. Such conversations may be provided by a chatbot, voice assistance agent, or other bot or assistant. For example, a chatbot conversational agent may receive and transmit a series of messages with respective medical users or patient users, as accessed via an agent data interface 506. The chatbot conversational agent may be accessed from a user messaging interface such as via a graphical user interface messaging interface 523 or 543 discussed below. Processing the data may include executing one or more other models (e.g., language or non-language models) or algorithms that are adapted to generate data (e.g. estimated battery life), with this data being provided as part of the input to the language model 502.

The care coordination system 500 communicates with the patient computing device 520 and/or patient programming device 530 via a network 510, including to obtain patient data that is used as background or ongoing context for operation of the AI/language processing models 502. The data may be stored in a database 504 or another large-scale data store (e.g., data lake) specifically for the patient or for a population of patients. The care coordination system 500 may also perform data analysis with processing engines (not shown) that parse and determine a patient state from device operation, program usage, medical records, etc. In some examples, other AI models or algorithms may be invoked to analyze neurostimulation usage, programs, particular parameters, patient actions, clinician actions, or health outcomes. Likewise, the care coordination system 500 may also perform data analysis on sensor data from one or more patient sensors (e.g., wearables, sleep trackers, motion trackers, implantable devices, etc.) in one or more internal or external devices.

In addition to producing content for natural language conversations and text output, the care coordination system 500 may evaluate natural language conversations and text input originating from the patient, caregiver, or medical user. Such conversations may be logged and maintained on an ongoing basis as part of chat session data 505 and used as input to the language model or the other models. For example, the chat session data 505 may be used to inform later parts of the same conversation or future conversations with the same or different users.

The care coordination system 500 may be accessed by a medical user computing device 540 to enable a doctor, medical device company representative, or other medical professional to start and join natural language conversations regarding neurostimulation outcomes and medical advice. A medical user computing device 540 may be a personal computer, tablet, smartphone, or other form of user-interactive device. The medical user computing device 540 hosts the graphical user interface 543 that provides input and output (e.g., in the form of messages) in a natural language conversation with a conversational agent of the care coordination system 500 (e.g., accessed via the agent data interface 506). The medical user computing device 540 may also include notification logic 541 to alert the medical user regarding a particular natural language conversation (e.g., where a medical user is requested or needs to provide input); and programming configuration logic 542 that is used to suggest, approve, and cause delivery of particular programming data values and operational settings to a patient or group of patients.

The care coordination system 500 may be accessed by a patient computing device 520, to enable a patient, caregiver, or other end user to start or join the natural language conversations regarding neurostimulation outcomes and medical advice. A patient computing device 520 may be a personal computer, tablet, smartphone, or other form of user-interactive device. The patient computing device 520 receives and provides interaction with the patient using a graphical user interface 523, implementing notification logic 521 and programming configuration logic 522. The graphical user interface 523 may provide a messaging interface (e.g., chat entry and display interface). For instance, the messaging interface may be used to receive input from a patient via freeform questionnaires, structured surveys, natural language messaging answers, or other inputs. Such inputs may provide text or other responses related to pain or satisfaction, the psychological or physiological state of the patient, neurostimulation treatment results, or related conditions.

In addition to coordinating natural language conversations, the care coordination system 500 may provide other therapy content and recommendations to the medical user computing device 540, patient computing device 520, or patient programming device 530. The patient programming device 530 is depicted as including a user interface 531 and program implementation logic 532. The program implementation logic 532 specifically may provide the patient with the ability to implement or switch to particular programs and recommendations such as those suggested in natural language conversations. In some examples, the patient programming device 530 directly receives programming recommendations or settings via a network. The patient programming device 530 communicates programming data 550 to the stimulation device 221 in the form of settings, programs, and data values. In other examples, instructions on how to implement the recommended programming settings are explained to the user (e.g., via the graphical user interface 523) and the user enters the settings manually into the patient programming device 530 via the user interface 531.

The care coordination system 500 may implement multiple algorithms, models, and/or agents, each of which may be specialized in handling different scenarios. The care coordination system 500 may utilize a variety of logic to determine how and when to escalate a particular messaging or conversational session to another human user. One example includes the use of a rules-based approach for escalating a conversation to add/include another user. For instance, the presence of certain trigger words, or conversation outcomes (e.g., becoming longer than a specific threshold without resolution) may cause the care coordination system 500 to escalate to a specific human user, such as a clinician who is familiar with the issue that the patient is experiencing. Another example includes the use of an AI-based approach, for identifying scenarios where human intervention may be needed. This AI-based approach may include monitoring for deviation from specific topics, identifying a lack of information in a knowledge base on which to base an answer, identifying a high-risk scenario (e.g., patient complains of very high pain level), etc.

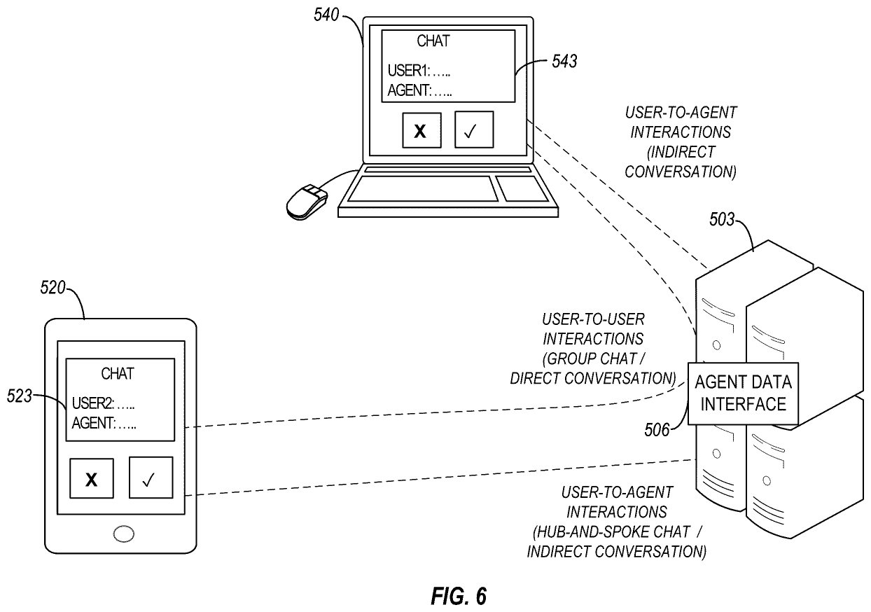

FIG. 6 depicts a scenario for coordinating natural language conversations between a patient user and a medical user using the care coordination system 500. Specifically, this depicts how the care coordination system 500 may be utilized with different communication models, to coordinate interactions in different ways. This figure shows how the graphical user interface 543 on a medical user computing device 540 provides a natural language conversation in a chat session, based on communications with agent data interface 506. This figure also shows how the graphical user interface 523 on a patient computing device 520 also provides another natural language conversation in a corresponding chat session.

A first example communication model used in this scenario is a group chat model, depicted in more detail in FIGS. 7, 8A, and 8B. With the group chat model, the care coordination system 500 handles multiple parties in a single chat conversation among multiple of the patient, medical device company representative (or medical device company agent), clinician, caregiver, and the AI agent. When needed, the care coordination system 500 coordinates conversations to provide participants with contextual summary information in a role-specific way. As one example, the care coordination system 500 may generate a summary text conversation of recent device usage challenges expressed by the patient, and provide this summary to a patient care representative who joins the in-progress conversation.

A second example communication model is a hub and spoke chat model, depicted in more detail in FIGS. 9, 10A, and 10B. With the hub and spoke chat model, the AI agent (AI agent 910) is the central mediator of messages between participants (patient, medical device company representative (or agent), clinician, caregiver) who do not directly chat with each other. In either communication model, the care coordination system 500 selectively allows information to be shown specific to roles to respect privacy or other concerns (e.g., with redacted information).

FIG. 7 depicts example user-based data flows, using the care coordination system 500 that includes an artificial intelligence (AI)-based conversational agent (a care coordination conversational agent 710). The care coordination system 500 is adapted to obtain relevant data from data sources 720. This data is used to customize and adapt natural language conversations via the care coordination conversational agent 710.

Here, the agent 710 performs natural language conversations with the medical user 730, shown as medical user-agent interactions 735 (e.g., interactions to send and receive natural language content at each party). The agent 710 also performs natural language conversations with the patient 740, shown as patient-agent interactions 745 (e.g., interactions to send and receive natural language content at each party).

Additionally, the agent 710 enables natural language conversations between the medical user 730 and the patient 740 within one or more conversations (e.g., chat sessions), shown as patient-medical user interactions 755. The patient-medical user interactions may begin based on escalation of some scenario in the conversation, such as where an AI agent cannot provide suitable assistance. Additional scenarios on how escalation may arise in a natural language conversation is depicted in more detail in FIGS. 8A and 8B.

FIG. 8A and FIG. 8B depict example user chat sessions and a corresponding flowchart of operations with the care coordination conversational agent 710. First, in FIG. 8A, a series of natural language conversation entries and responses are entered by two entities: the patient 740 and the AI agent 710. For instance, the AI agent 710 provides responses 801, 802, and 803 to conversational information provided by the patient, and the patient 740 provides responses 811 and 812 to specific questions provided by the AI agent 710.

Based on the content of the patient responses 811 and 812, a determination is made that escalation to another user is required. FIG. 8A shows how a third entity is brought into the conversation after response 812, in response to determining a particular state or condition of the neurostimulation (e.g., that a neurostimulation program is not working). The AI agent generates a response 803 that explains how the third entity will be brought into the conversation, along with an example explanation/summary of the conversation. A reply from the third entity—a medical device company representative—is introduced at response 821. Accordingly, assistance from the third entity can be seamlessly introduced into the ongoing natural language conversation.

Next, FIG. 8B depicts a sequence of conversational operations, illustrating how AI responses versus user inputs are provided in the natural language conversation with a chatbot. First, the AI response (response 802, depicted in FIG. 8A) is provided, followed by the patient input response (response 812, depicted in FIG. 8A). This response-input sequence potentially repeats in the chatbot until a determination 831 of an escalation. This determination may be based on specific keywords, semantic meaning of patient input, or contextual evaluation of a patient's medical state. This determination may be performed by a care coordination agent 830 (e.g., an implementation of AI agent 710), to coordinate information among multiple users in an escalation condition.

If an escalation condition is detected at determination 831, then an escalated AI response (response 803, depicted in FIG. 8A) is provided, followed by a summary 805 of the chat to be provided to a third party (e.g., a medical device company representative). The representative may be notified by one or more electronic communications (e.g., by an email, SMS message, app notification, etc.), and receive this summary 805.

The remainder of the flow of FIG. 8B depicts the outcomes of escalating to the third party, with the addition of patient-medical user interactions 755 into the chat session. A determination is made at decision 841 based on whether the medical device company representative joins the chat. If the agent does not join the chat, then an AI exit response may be provided with an alternate workflow message 807 (e.g., a response that states, “Let's schedule a time to talk to your medical device company representative. Would any of the following times work?”). If the agent does join the chat, then the response 821 may be received from the representative and entered into the chat session.

Additional interaction may occur from a patient input, such as is provided in response 813. This may be a complex response (e.g., asking questions to the representative, providing more details to the representative), or a simple response (e.g., Yes or No answers, or “Thank you for your help”). The chatbot may provide additional responses and content based on the context of the chat session, such as providing an exit response 808 providing a summary of the outcome (e.g., “Here's a summary of our chat today . . . ”).

FIG. 9 depicts example agent-coordinated data flows, using a care coordination system that includes an artificial intelligence (AI)-based conversational agent (a care coordination conversational agent 910). The care coordination system 500 is also adapted to obtain relevant data from data sources 900. This data is used to customize and adapt natural language conversations via the care coordination conversational agent 910.

Here, the agent 910 performs natural language conversations with any number of users, including a clinician 930, a caregiver 940, a patient 950, and a medical device company representative 960. Unlike FIG. 7, all of the interactions among parties are directly coordinated in separate conversations with the AI agent 910. This is shown in the form of clinician-agent interactions 935, caregiver-agent interactions 945, patient-agent interactions 955, and rep-agent interactions 965.

FIG. 10A and FIG. 10B depict additional example agent chat sessions and a corresponding flowchart of operations with the care coordination conversational agent 910. First, in FIG. 10A, a series of natural language conversation entries and responses are entered by four entities: the patient 950, the clinician 930, the medical device company representative 960, and the AI agent 910. Each of these conversations are separately conducted, but content in the conversations is coordinated.

For instance, in a first conversation 1010, the AI agent 910 provides responses 1001, 1002, 1003, 1004, and 1005 to conversational information provided by the patient, and the patient 950 provides responses 1011, 1012, 1013 to specific questions provided by the AI agent 910. One of these responses, response 1003, indicates that the AI agent will notify the medical device company representative and a supervising physician.

The outcome of notifying the medical device company representative is shown in a second conversation 1020. Here, the AI agent first provides a response 1021 to communicate informative information to the medical device company representative; and the medical device company representative provides a response 1031 with an instruction to the agent.

The outcome of notifying the physician is shown in a third conversation 1030. Here, the AI agent first provides a response 1041 to communicate informative information to the physician, followed by a response 1042 which indicates what action or recommendation has been provided by the medical device company representative. The physician provides a response 1051 with an inquiry (question), and the AI agent provides another response 1042 with specific information regarding program usage. The physician provides another response 1052 with an instruction, related to specific neurostimulation actions to be implemented and suggested to the patient.

Next, FIG. 10B depicts a sequence of conversational operations, illustrating how AI responses versus user inputs are provided in the respective natural language conversations with a chatbot. First, the AI response (response 1002, depicted in FIG. 10A) is provided, followed by the patient input response (response 1012, depicted in FIG. 10A). This response-input sequence potentially repeats in the chatbot until a determination 1061 of an escalation. As noted above, this determination may be based on specific keywords, semantic meaning of patient input, or contextual evaluation of a patient's medical state. This determination may be performed by a care coordination agent 1060 (e.g., an implementation of AI agent 910), to coordinate separate conversations among multiple users in an escalation condition.

If an escalation condition is detected at determination 1061, then an escalated AI response (response 1003, depicted in FIG. 10A) is provided, followed by an AI agent coordination 1062 to initiate contact 1063 with a medical device company representative and to initiate contact 1064 with a physician. As above, a notification to request the additional party's involvement can be provided by one or more electronic communications (e.g., by an email, SMS message, app notification, etc.).

The involvement of a medical device company representative is shown at the center of FIG. 10B, and includes an agent summary (response 1021, depicted in FIG. 10A) and input from the medical device company representative (response 1031, depicted in FIG. 10A). The involvement of a physician is shown at the right of FIG. 10B, and includes an agent summary (response 1041, depicted in FIG. 10A) and input from the physician (response 1051, depicted in FIG. 10A). Based on these involvements, additional AI responses may be provided (such as responses 1004, 1042, and 1043 depicted in FIG. 10A).

Additional interaction may occur from a patient input, such as is provided in response 1014. This may be a complex response (e.g., asking questions to the representative or the physician, providing more details in response to a specific question asked by either), or a simple response (e.g., Yes or No answers, or “Thank you for your help”). The chatbot may provide additional responses and content based on the context of the chat session, such as providing an exit response 1006 providing a summary of the outcome (e.g., “Here's a summary of our chat today and actions that were recommended . . . ”).

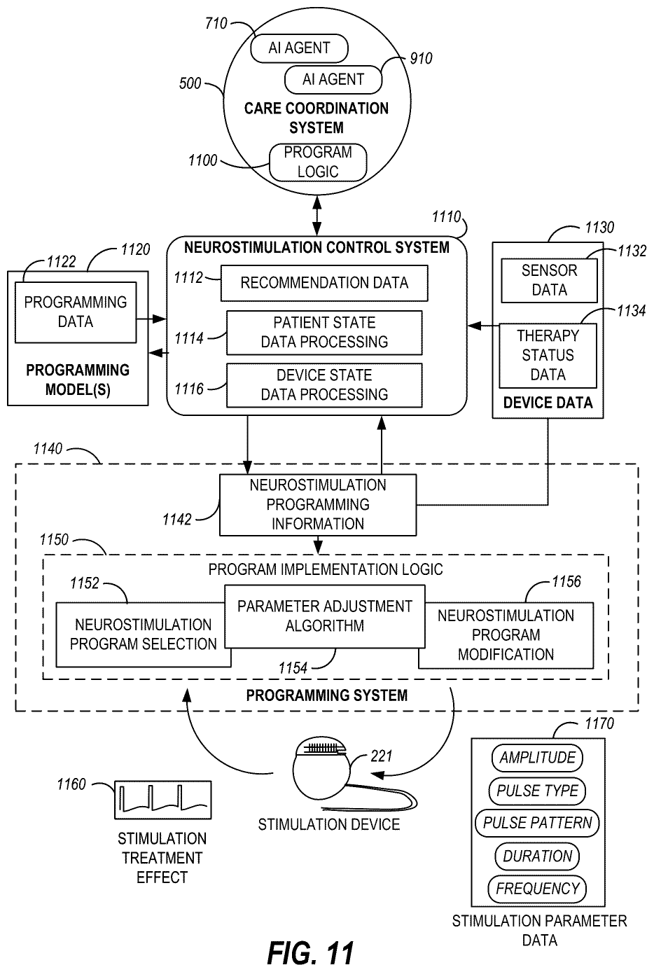

FIG. 11 illustrates, by way of example, an embodiment of a data processing flow for programming changes for neurostimulation treatments in a human patient, in connection with use of the care coordination system 500 or other coordination with natural language conversations. Specifically, this data processing flow shows how a neurostimulation control system 1110 may cause a change to neurostimulation settings/programming of a stimulation device 221, in response to natural language conversations performed in the care coordination system 500 and specifically via AI agents 710, 910.

In this example, the care coordination system 500 may operate the AI agent 710 (coordinating user-to-user conversations) or the AI agent 910 (coordinating centralized multi-party conversations) as discussed above, to collect language conversations related to neurostimulation outcomes (e.g., issues, problems, benefits, etc.). The results of these conversations may be analyzed by program logic 1100 in the care coordination system 500. The program logic 1100 may determine scenarios where programming changes are required or suggested. This program logic 1100 may also receive instructions from authorized medical users (e.g., to disable, enable, change, or deploy some programming, as indicated in a natural language conversation).

The neurostimulation control system 1110 performs patient state data processing 1114 and device state data processing 1116 functions, to generate suggestions or recommendations with recommendation data 1112 (e.g., recommended programming settings). The recommendation data 1112 can be generated based on programming data 1122 produced by a trained programming model 1120, which itself may be a trained AI model. User adoption of the recommendations, in turn, will result in updated neurostimulation programming information 1142 that modifies the operation of the stimulation device 221. Such recommendations may be automatically authorized or manually approved, e.g., in the natural language conversations performed with AI agents 710 and 910.

FIG. 11 also depicts the evaluation of device data 1130, such as sensor data 1132, therapy status data 1134, and other treatment aspects that may be obtained or derived from the stimulation device 221 or related neurostimulation programming and device operation. The device data 1130 can be further evaluated with patient state data processing 1114 and device state data processing 1116, to produce patient-specific programming recommendations and patient state-specific programming recommendations. As will be understood, the recommendations for the same patient may be different at separate points in time, based on the patient's current state. In addition to programming recommendations, non-programming recommendations and related content (e.g., suggestions, guidance, instructions) may be selected and provided to the user. Non-programming recommendations may relate to device operation aspects such as, how to efficiently charge a neurostimulation device, how to use a remote control to control programming, etc. Non-programming recommendations may also integrate general suggestions related to health recommendations or guidance, such as how to manage pain in general, with use of the device or based on therapeutic activities and actions.

The inputs considered by the care coordination system 500 (e.g., as a result of natural language conversations) can be used to refine or customize the recommendations that are generated, such as to change the type or aspects of the recommendations. The trained programming model 1120 may generate new or updated programming data 1122 (e.g., programs, program parameter values) based on evaluation of the patient state data processing 1114, device state data processing 1116, and similar patient or clinician data.

The remainder of the data processing flow illustrates how the neurostimulation control system 1110 implements programming, including in a closed-loop (or partially-closed-loop) system, which is adapted based on natural language conversations in the care coordination system 500. A programming system 1140 uses programming information 1142 provided from the neurostimulation control system 1110 as an input to program implementation logic 1150. The program implementation logic 1150 may be implemented by a parameter adjustment algorithm 1154, which affects a neurostimulation program selection 1152 or a neurostimulation program modification 1156 (e.g., with such a program selection or modification directly caused by a recommendation). For instance, some parameter changes may be implemented by a simple modification to a program operation; other parameter changes may require a new program to be deployed. The results of the parameter or program changes or selection provide various stimulation parameters 1170 to the stimulation device 221, causing a different or new stimulation treatment effect 1160.

By way of example, operational parameters of the stimulation device that may be generated, identified, or evaluated by the neurostimulation control system 1110 may include amplitude, frequency, duration, pulse width, pulse type, patterns of neurostimulation pulses, waveforms in the patterns of pulses, and like settings with respect to the intensity, type, and location of neurostimulator output on individual or a plurality of respective leads. The neurostimulator may use current or voltage sources to provide the neurostimulator output, and apply any number of control techniques to modify the electrical simulation applied to anatomical sites or systems related to pain or analgesic effect. In various embodiments, a neurostimulator program may be defined or updated to indicate parameters that define spatial, temporal, and informational characteristics for the delivery of modulated energy, including the definitions or parameters of pulses of modulated energy, waveforms of pulses, pulse blocks each including a burst of pulses, pulse trains each including a sequence of pulse blocks, train groups each including a sequence of pulse trains, and programs of such definitions or parameters, each including one or more train groups scheduled for delivery. Characteristics of the waveform that are defined in the program may include, but are not limited to the following: amplitude, pulse width, frequency, total charge injected per unit time, cycling (e.g., on/off time), pulse shape, number of phases, phase order, interphase time, charge balance, ramping, as well as spatial variance (e.g., electrode configuration changes over time). It will be understood that based on the many characteristics of the waveform itself, a program may have many parameter setting combinations that would be potentially available for use.

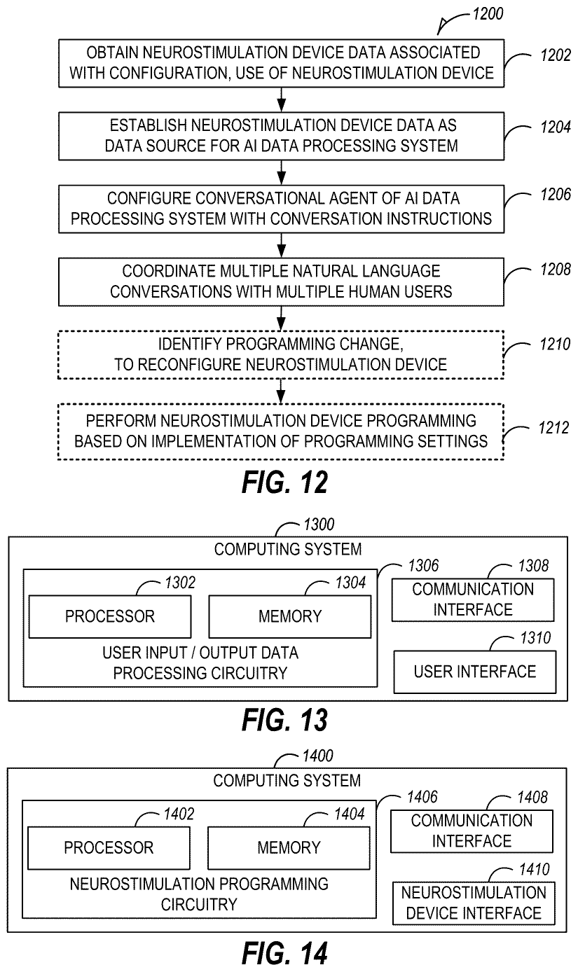

FIG. 12 illustrates, by way of example, an embodiment of a method 1200 implemented by a system or device configured to enable coordination of medical information among multiple users associated with a neurostimulation device, in connection with a care coordination system and natural language conversational agents. For example, the method 1200 can be embodied by electronic operations performed by one or more computing systems or devices (including those at a network-accessible remote service) that are specially programmed to implement data analysis and/or natural language data processing operations. In specific examples, the operations of the method 1200 may be implemented through the systems and data flows depicted above, at a single entity or at multiple locations.

In an example, the method 1200 begins at 1202 by obtaining neurostimulation device data associated with the configuration and the use of a neurostimulation device (e.g., an implantable neurostimulation device in use at a patient, which is programmed and operable for treatment of some particular medical condition of the patient). In an optional example, the method 1200 also includes obtaining patient data associated with the particular medical condition to be treated by the neurostimulation device, such as patient medical records, or treatment or medical history. In still further examples, other data sources may provide data that is not directly related to the particular medical condition to be treated by the neurostimulation device, such as activity or health data obtained from wearables, environment data (e.g. weather data), and the like.

The method 1200 continues at 1204 by establishing neurostimulation device data as a data source for AI data processing system (e.g., a care coordination system 500) as discussed above. This may include providing the neurostimulation device data as a data source to an implementation or a configuration of the AI data processing system that includes a pre-trained model and an agent to interact with the pre-trained model. In an example, the pre-trained model includes one or more LLMs, and the agent is a conversational agent (e.g., chatbot or voice agent). The AI data processing system may use other types of NLP engines or language production subsystems in addition or in place of the one or more LLMs. In further examples, the patient data (e.g., associated with the particular medical condition to be treated by the neurostimulation device) is provided as another data source for the AI data processing system.

The method 1200 continues at 1206 by configuring the conversational agent of the AI data processing system with conversation instructions (e.g., specific prompts or directives that are interpreted and processed by the LLM to adapt the type of data processing and output provided by the conversational agent). In an example, the conversation instructions cause the conversational agent to perform natural language conversations with respective human users based on the configuration and use of the neurostimulation device. In another example, the conversation instructions cause the conversational agent to perform the natural language conversations with the respective human users based on the medical condition to be treated by the neurostimulation device, or other aspects of the patient data, activity data, health data, environmental data, etc. related to the medical condition to be treated.

The conversation instructions may also configure specific actions to be taken by the conversational agent or by the AI data processing system. For instance, the conversational agent may identify a special operational condition related to the configuration and use of the neurostimulation device (e.g., some urgent problem or medical condition), based on the content of the ongoing conversations. The conversational agent may be configured and operated the agent to provide a notification of the special operational condition in the ongoing conversations, provide a notification to other users not currently involved in the ongoing conversations, or trigger/cause other notifications or alerts.

The method 1200 continues at 1208 by operating the conversational agent to coordinate multiple natural language conversations with multiple human users, e.g., a first human user and a second human user. The first human user may be a patient using the neurostimulation device, and the second human user may be a user associated with care of the patient (e.g. a caregiver or a medical user such as a physician, company representative, or other medical agent). In a specific example, the content in the multiple, ongoing natural language conversations is coordinated by: conducting the first natural language conversation between the first human user and the conversational agent; identifying a condition for conversation coordination, based on content in the first natural language conversation or the second natural language conversation; conducting the second natural language conversation between the second human user and the agent; and joining the second human user into the first natural language conversation. This scenario is described in more detail in the examples of FIGS. 8A and 8B, above.

In another specific example, the content in the multiple, ongoing natural language conversations is coordinated by: conducting the first natural language conversation between the first human user and the agent; conducting the second natural language conversation between the first human user and the agent; identifying a condition for conversation coordination, based on content in the first natural language conversation; modifying the first natural language conversation based on content from the second natural language conversation, as managed by the agent; and modifying the second natural language conversation based on content from the first natural language conversation, as managed by the agent. This scenario is described in more detail in the examples of FIGS. 10A and 10B, above.

In another specific example, the content in the multiple, ongoing natural language conversations is coordinated among three or more users. The conversational agent may be operated to coordinate the content of the first natural language conversation and the content of the second natural language conversation with a third natural language conversation that occurs between the conversational agent and a third human user. Other examples may include: involvement with additional users; the output of the conversations in an interface designed for use in a patient device, such as a smartphone operable by a patient; the output of the conversations in an interface designed for use in a medical user device, such as a computing device operable by a medical user or an assistive user (e.g., caregiver or human agent);

In further examples, the conversational agent selects and provides additional information to the respective human users, such as in contexts where the conversational agent generates and outputs recommendations or suggested content for the patient. As a first example, the recommendations for the patient may relate to the configuration and use of the neurostimulation device (e.g., regarding usage of particular programs, remote control use, etc.). As a second example, the recommendations for the patient may provide information related to a medical condition to be treated by the neurostimulation device (e.g. general guidance on managing the patient's condition). Such recommendations can be output in a natural language conversation occurring between the conversational agent and the patient. The recommendations may be summarized and provided to the other human users (such as a supervising or coordinating medical user).

In other further examples, the historical conversation data of the conversational agent is maintained (e.g., stored) and provided as another data source for the AI data processing system. Such historical conversation data can include or be based on previous natural language conversations provided between the respective human users and the agent. Accordingly, the conversational agent can conduct the natural language conversations with the respective human users based on the contents, instructions, prompts, and commands exchanged in the historical conversation data.

As a specific example, the AI data processing system may maintain and use conversation history and details when coordinating the respective conversations. For instance, the AI data processing system may record a conversation history of a first natural language conversation occurring between the agent and the first human user and of a second natural language conversation occurring between the agent and the second human user.

In further examples, the method 1200 continues at 1210 by optionally identifying one or more programming change(s), to configure the neurostimulation device for treatment. The programming change(s) may be related to the configuration and use of the neurostimulation device, and may be identified by the AI data processing system based on the content of the first natural language conversation or the content of the second natural language conversation (and related conversation or patient data). The programming change(s) may be directly identified and implemented, or provided as programming change recommendation(s) to the patient or medical user.

In further examples, the method 1200 continues at 1212 by optionally performing neurostimulation device programming based on implementation of the programming settings. Such programming may occur in connection with the use of a program, and the additional collection and evaluation of device data during use of the program that performs the neurostimulation treatment. As non-limiting examples, the data values that reconfigure the neurostimulation device may cause a change to one or more of: timing, amplitude, frequency, intensity, duration, pulse patterns, pulse shapes, a spatial location of pulses, waveform shapes, or a spatial location of waveform shapes, of modulated energy provided with a plurality of leads of the neurostimulation device. Other aspects of device programming and feedback may be provided within or subsequent to the method 1200.

FIG. 13 illustrates, by way of example, a block diagram of an embodiment of a system 1300 (e.g., a computing system) for performing analysis of patient data, configuring and operating a conversational agent, and implementing neurostimulation programming changes in connection with the data coordination and conversational data processing operations discussed above. The system 1300 may be integrated with or coupled to a computing device, a remote control device, patient programmer device, clinician programmer device, program modeling system, or other external device, deployed with neurostimulation treatment. In some examples, the system 1300 may be a networked device (server) connected via a network (or combination of networks) which communicates to one or more devices (clients) using a communication interface 1308 (e.g., communication hardware which implements software network interfaces and services). The network may include local, short-range, or long-range networks, such as Bluetooth, cellular, IEEE 802.11 (Wi-Fi), or other wired or wireless networks.

The system 1300 includes a processor 1302 and a memory 1304, which can be optionally included as part of user input/output data processing circuitry 1306. The processor 1302 may be any single processor or group of processors that act cooperatively. The memory 1304 may be any type of memory, including volatile or non-volatile memory. The memory 1304 may include instructions, which when executed by the processor 1302, cause the processor 1302 to implement data processing, or to enable other features of the user input/output data processing circuitry 1306. Thus, electronic operations in the system 1300 may be performed by the processor 1302 or the circuitry 1306.

For example, the processor 1302 or circuitry 1306 may implement any of the features of the method 1200 (such as operations 1202-1210) to obtain and process patient data, to operate a conversational agent and an AI data processing system, to coordinate natural language conversations with multiple human users, to identify programming changes based on the natural language conversations, and to provide programming settings that implement the identified programming changes. It will be understood that the processor 1302 or circuitry 1306 may also implement other aspects of the logic and processing described above, as adapted for closed-loop (or partially-closed-loop) device programming, re-programming, or device programming recommendation activities.

FIG. 14 illustrates, by way of example, a block diagram of an embodiment of a system 1400 (e.g., a computing system) implementing neurostimulation programming circuitry 1406 to cause programming of an implantable electrical neurostimulation device, for accomplishing the therapy objectives in a human subject based on the closed-loop recommendations discussed herein. The system 1400 may be operated by a clinician, a patient, a caregiver, a medical facility, a research institution, a medical device manufacturer or distributor, and embodied in a number of different computing platforms. The system 1400 may be a remote control device, patient programmer device, program modeling system, or other external device, including a regulated device used to directly implement programming commands and modification with a neurostimulation device. In some examples, the system 1400 may be a networked device connected via a network (or combination of networks) to a computing system operating a user interface computing system using a communication interface 1408. The network may include local, short-range, or long-range networks, such as Bluetooth, cellular, IEEE 802.11 (Wi-Fi), or other wired or wireless networks.

The system 1400 includes a processor 1402 and a memory 1404, which can be optionally included as part of neurostimulation programming circuitry 1406. The processor 1402 may be any single processor or group of processors that act cooperatively. The memory 1404 may be any type of memory, including volatile or non-volatile memory. The memory 1404 may include instructions, which when executed by the processor 1402, cause the processor 1402 to implement the features of the neurostimulation programming circuitry 1406. Thus, the electronic operations in the system 1400 may be performed by the processor 1402 or the circuitry 1406.

The processor 1402 or circuitry 1406 may directly or indirectly implement neurostimulation operations associated with the method 1200, including neurostimulation device programming based on recommendations or actions discussed in the natural language conversations (to implement operations 1210, 1212). The processor 1402 or circuitry 1406 may further provide data and commands to assist the processing and implementation of the programming using communication interface 1408 or a neurostimulation device interface 1410. It will be understood that the processor 1402 or circuitry 1406 may also implement other aspects of the device data processing or device programming functionality described above.

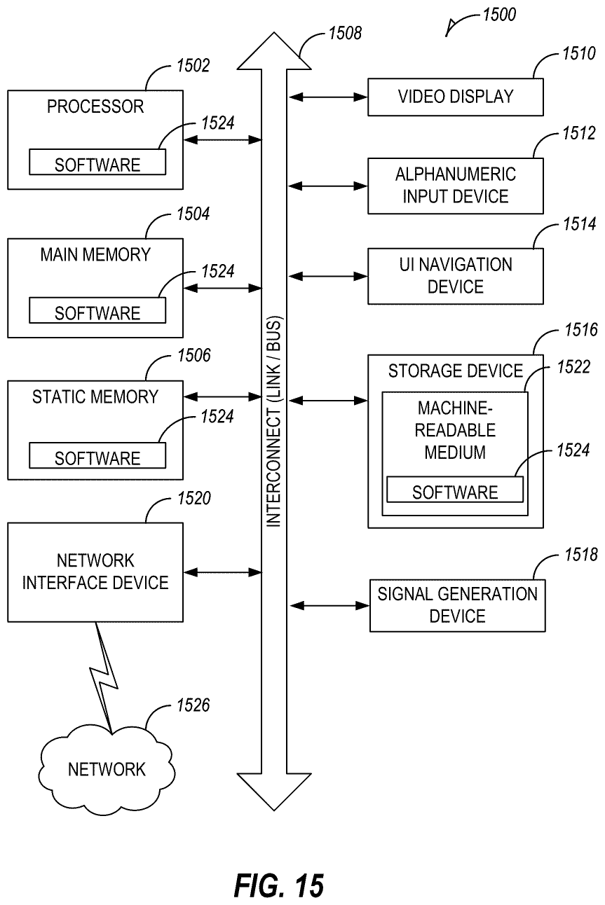

FIG. 15 is a block diagram illustrating a machine in the example form of a computer system 1500, within which a set or sequence of instructions may be executed to cause the machine to perform any one of the methodologies discussed herein, according to an example embodiment. In alternative embodiments, the machine operates as a standalone device or may be connected (e.g., networked) to other machines. In a networked deployment, the machine may operate in the capacity of either a server or a client machine in server-client network environments, or it may act as a peer machine in peer-to-peer (or distributed) network environments. The machine may be a personal computer (PC), a tablet PC, a hybrid tablet, a personal digital assistant (PDA), a mobile telephone, an implantable pulse generator (IPG), an external remote control (RC), a User's Programmer (CP), or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. Similarly, the term “processor-based system” shall be taken to include any set of one or more machines that are controlled by or operated by a processor (e.g., a computer) to individually or jointly execute instructions to perform any one or more of the methodologies discussed herein.

Example computer system 1500 includes at least one processor 1502 (e.g., a central processing unit (CPU), a graphics processing unit (GPU) or both, processor cores, compute nodes, etc.), a main memory 1504 and a static memory 1506, which communicate with each other via a link 1508 (e.g., bus). The computer system 1500 may further include a video display unit 1510, an alphanumeric input device 1512 (e.g., a keyboard), and a user interface (UI) navigation device 1514 (e.g., a mouse). In one embodiment, the video display unit 1510, input device 1512 and UI navigation device 1514 are incorporated into a touch screen display. The computer system 1500 may additionally include a storage device 1516 (e.g., a drive unit), a signal generation device 1518 (e.g., a speaker), a network interface device 1520, and one or more sensors (not shown), such as a global positioning system (GPS) sensor, compass, accelerometer, or other sensor. It will be understood that other forms of machines or apparatuses (such as PIG, RC, CP devices, and the like) that are capable of implementing the methodologies discussed in this disclosure may not incorporate or utilize every component depicted in FIG. 15 (such as a GPU, video display unit, keyboard, etc.).

The storage device 1516 includes a machine-readable medium 1522 on which is stored one or more sets of data structures and instructions 1524 (e.g., software) embodying or utilized by any one or more of the methodologies or functions described herein. The instructions 1524 may also reside, completely or at least partially, within the main memory 1504, static memory 1506, and/or within the processor 1502 during execution thereof by the computer system 1500, with the main memory 1504, static memory 1506, and the processor 1502 also constituting machine-readable media.

While the machine-readable medium 1522 is illustrated in an example embodiment to be a single medium, the term “machine-readable medium” may include a single medium or multiple media (e.g., a centralized or distributed database, and/or associated caches and servers) that store the one or more instructions 1524. The term “machine-readable medium” shall also be taken to include any tangible (e.g., non-transitory) medium that is capable of storing, encoding or carrying instructions for execution by the machine and that cause the machine to perform any one or more of the methodologies of the present disclosure or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

The instructions 1524 may further be transmitted or received over a communications network 1526 using a transmission medium via the network interface device 1520 utilizing any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Wi-Fi, 3G, and 4G LTE/LTE-A or 5G networks). The term “transmission medium” shall be taken to include any intangible medium that is capable of storing, encoding, or carrying instructions for execution by the machine, and includes digital or analog communications signals or other intangible medium to facilitate communication of such software.

The above detailed description is intended to be illustrative, and not restrictive. The scope of the disclosure should, therefore, be determined with references to the appended claims, along with the full scope of equivalents to which such claims are entitled.

What is claimed is:

1. A method for enabling coordination of medical information among multiple users associated with a neurostimulation device, comprising:
obtaining neurostimulation device data associated with configuration and use of the neurostimulation device;
providing the neurostimulation device data as a data source for an artificial intelligence (AI) data processing system, the AI data processing system including a pre-trained model and an agent to interact with the pre-trained model;
configuring the agent with conversation instructions, the conversation instructions to cause the agent to perform natural language conversations with respective human users based on the configuration and use of the neurostimulation device; and
operating the agent to coordinate content of a first natural language conversation that occurs between the agent and a first human user with content of a second natural language conversation that occurs between the agent and a second human user.

2. The method of claim 1, wherein the pre-trained model includes a large language model (LLM), wherein the first human user is a patient using the neurostimulation device, and wherein the second human user is a user associated with care of the patient.

3. The method of claim 2, further comprising:
operating the agent to generate recommendations for the patient, wherein the recommendations for the patient relate to: (i) the configuration and use of the neurostimulation device, or (ii) information related to a medical condition to be treated by the neurostimulation device; and
outputting the recommendations in the first natural language conversation occurring between the agent and the patient.

4. The method of claim 1, wherein to coordinate the content of the first natural language conversation with the content of the second natural language conversation includes:
conducting the first natural language conversation between the first human user and the agent;
identifying a condition for conversation coordination, based on content in the first natural language conversation or the second natural language conversation;
conducting the second natural language conversation between the second human user and the agent; and
joining the second human user into the first natural language conversation.

5. The method of claim 1, wherein to coordinate the first natural language conversation with the second natural language conversation includes:
conducting the first natural language conversation between the first human user and the agent;
conducting the second natural language conversation between the first human user and the agent;
identifying a condition for conversation coordination, based on content in the first natural language conversation;
modifying the first natural language conversation based on content from the second natural language conversation, as managed by the agent; and
modifying the second natural language conversation based on content from the first natural language conversation, as managed by the agent.

6. The method of claim 1, further comprising:
operating the agent to coordinate the content of the first natural language conversation and the content of the second natural language conversation with a third natural language conversation that occurs between the agent and a third human user.

7. The method of claim 1, further comprising:
identifying a special operational condition related to the configuration and use of the neurostimulation device, based on the content of the first natural language conversation or the content of the second natural language conversation; and
operating the agent to provide a notification of the special operational condition in the first natural language conversation or the second natural language conversation.

8. The method of claim 1, further comprising:
obtaining patient data associated with a medical condition to be treated by the neurostimulation device; and
providing the patient data as another data source for the AI data processing system;
wherein the conversation instructions are further to cause the agent to perform the natural language conversations with the respective human users based on the medical condition.

9. The method of claim 1, further comprising:
maintaining historical conversation data of the agent as another data source for the AI data processing system, wherein the historical conversation data is based on previous natural language conversations provided between the respective human users and the agent;
wherein the conversation instructions are further to cause the agent to perform the natural language conversations with the respective human users based on the historical conversation data.

10. The method of claim 1, further comprising:
identifying a programming change related to the configuration and use of the neurostimulation device, based on the content of the first natural language conversation or the content of the second natural language conversation; and
transmitting a command, based on the identified programming change, to reconfigure one or more programming data values of the neurostimulation device;
wherein the command to reconfigure the one or more programming data values of the neurostimulation device causes a change to one or more of: timing, amplitude, frequency, intensity, duration, pulse patterns, pulse shapes, a spatial location of pulses, waveform shapes, or a spatial location of waveform shapes, of modulated energy provided with a plurality of leads of the neurostimulation device.

11. A computing system to enable coordination of medical information among multiple users associated with a neurostimulation device, the computing system comprising:
one or more processors; and
one or more memory devices comprising instructions, which when executed by the one or more processors, cause the one or more processors to:
obtain neurostimulation device data associated with configuration and use of the neurostimulation device;
provide the neurostimulation device data as a data source for an artificial intelligence (AI) data processing system, the AI data processing system including a pre-trained model and an agent to interact with the pre-trained model;
configure the agent with conversation instructions, the conversation instructions to cause the agent to perform natural language conversations with respective human users based on the configuration and use of the neurostimulation device; and
operate the agent to coordinate content of a first natural language conversation that occurs between the agent and a first human user with content of a second natural language conversation that occurs between the agent and a second human user.

12. The computing system of claim 11, wherein the pre-trained model includes a large language model (LLM), wherein the first human user is a patient using the neurostimulation device, and wherein the second human user is a user associated with care of the patient.

13. The computing system of claim 12, wherein the instructions further cause the one or more processors to:
operate the agent to generate recommendations for the patient, wherein the recommendations for the patient relate to: (i) the configuration and use of the neurostimulation device, or (ii) information related to a medical condition to be treated by the neurostimulation device; and
output the recommendations in the first natural language conversation occurring between the agent and the patient.

14. The computing system of claim 11, wherein to coordinate the content of the first natural language conversation with the content of the second natural language conversation includes:
conducting the first natural language conversation between the first human user and the agent;
identifying a condition for conversation coordination, based on content in the first natural language conversation or the second natural language conversation;
conducting the second natural language conversation between the second human user and the agent; and
joining the second human user into the first natural language conversation.

15. The computing system of claim 11, wherein to coordinate the first natural language conversation with the second natural language conversation includes:
conducting the first natural language conversation between the first human user and the agent;
conducting the second natural language conversation between the first human user and the agent;
identifying a condition for conversation coordination, based on content in the first natural language conversation;
modifying the first natural language conversation based on content from the second natural language conversation, as managed by the agent; and
modifying the second natural language conversation based on content from the first natural language conversation, as managed by the agent.

16. The computing system of claim 11, wherein the instructions further cause the one or more processors to:
operate the agent to coordinate the content of the first natural language conversation and the content of the second natural language conversation with a third natural language conversation that occurs between the agent and a third human user.

17. The computing system of claim 11, wherein the instructions further cause the one or more processors to:
identify a special operational condition related to the configuration and use of the neurostimulation device, based on the content of the first natural language conversation or the content of the second natural language conversation; and
operate the agent to provide a notification of the special operational condition in the first natural language conversation or the second natural language conversation.

18. The computing system of claim 11, wherein the instructions further cause the one or more processors to:
obtain patient data associated with a medical condition to be treated with the neurostimulation device; and
provide the patient data as another data source for the AI data processing system;
wherein the conversation instructions are further to cause the agent to perform the natural language conversations with the respective human users based on the medical condition.

19. The computing system of claim 11, wherein the instructions further cause the one or more processors to:
maintain historical conversation data of the agent as another data source for the AI data processing system, wherein the historical conversation data is based on previous natural language conversations provided between the respective human users and the agent;
wherein the conversation instructions are further to cause the agent to perform the natural language conversations with the respective human users based on the historical conversation data.

20. The computing system of claim 11, wherein the instructions further cause the one or more processors to:
identify a programming change related to the configuration and use of the neurostimulation device, based on the content of the first natural language conversation or the content of the second natural language conversation; and
transmit a command, based on an identified programming change, to reconfigure one or more programming data values of the neurostimulation device;
wherein the command to reconfigure the one or more programming data values of the neurostimulation device causes a change to one or more of: timing, amplitude, frequency, intensity, duration, pulse patterns, pulse shapes, a spatial location of pulses, waveform shapes, or a spatial location of waveform shapes, of modulated energy provided with a plurality of leads of the neurostimulation device.
